(19) United States
(12) Patent Application Publication
(10) Pub. No.: US 2023/0200743 A1
(43) Pub. Date: Jun. 29, 2023

(54) APPARATUS AND METHOD FOR DIFFERENTIATING BETWEEN SYMPTOMS OF HEART ATTACK AND GERD

(71) Applicant: Micron Technology, Inc., Boise, ID (US)

(72) Inventors: Mandy W. FORTUNATI, Boise, ID (US); Kathryn H. RUSSO, Boise, ID (US); Srinivasa Anuradha BULUSU, Boise, ID (US)

(73) Assignee: Micron Technology, Inc., Boise, ID (US)

(21) Appl. No.: 17/560,926

(22) Filed: Dec. 23, 2021

(51) Int. Cl. A61B 5/00; A61B 10/00; A61B 5/145; A61B 5/15

(52) U.S. Cl. CPC A61B 5/7275; A61B 10/0051; A61B 5/743; A61B 5/14546; A61B 5/14507; A61B 5/1405

(57) ABSTRACT

An apparatus and method for testing a patient for distinguishing symptoms of a heart attack and GERD. A saliva specimen holder is configured to collect a saliva sample. A blood specimen holder with a well configured to collect a blood sample, a D-dimer testing zone, and a membrane in fluid communication with the well and the D-dimer testing zone. The membrane is configured to fluidly communicate a blood sample to the D-dimer testing zone. A composition on the membrane in the D-dimer testing zone is configured, when mixed with blood containing a D-dimer protein, to provide an indicator. A processor is programmed to analyze the collected blood sample to determine whether a D-dimer protein is detected in the blood sample and to analyze the saliva sample and to send information to the display indicating whether or not pepsin was detected.

### FIELD OF THE INVENTION

The present invention is directed to an apparatus and method for differentiating between symptoms of a heart attack and GERD and, more particularly, in one embodiment, to a testing device for permitting quick diagnosis between a heart attack and GERD.

### BACKGROUND

The cause of chest pain can be difficult to diagnosis. In a most critical situation, chest pain can be a sign that a person is having a heart attack. Heart attacks occur when the blood supply to the heart muscles becomes completely blocked. If a person does not receive immediate treatment, part of the heart muscle can die. However, chest pain can also be a symptom of less serious conditions, such as gastroesophageal reflux disease, commonly referred to as GERD.

A common symptom of a heart attack is pain or discomfort that typically occurs in the center or left side of the chest. This pain may come and go, and its severity can range from mild to severe. Other symptoms of a heart attack include intense pressure or tightness in the center of the chest; a feeling of heaviness or weakness in one or both arms; pain, numbness, or a tingling sensation in the arms, neck, jaw, lips, or stomach; difficulty breathing or shortness of breath; nausea and vomiting; dizziness or lightheadedness; fatigue and breaking out in a cold sweat. However, not everyone who has a heart attack experiences chest pain and the symptoms of a heart attack can vary considerably from person to person, and they may come on slowly or very suddenly.

According to one 2014 study, women having a heart attack are more likely to be misdiagnosed because they often don't experience the classic symptom of chest pain.

The symptoms of a heart attack are sometimes confused with heartburn. Heartburn is a symptom of acid reflux and GERD. Acid reflux occurs when acid from the stomach leaks up into the food pipe, or esophagus. One of the most common symptoms of acid reflux is heartburn, which is a painful burning sensation in the center of the chest just behind the breastbone, or sternum. GERD encompasses an array of disorders unified by the reflux of gastric contents. A person who experiences acid reflux more than twice a week for a few weeks may have GERD. According to one study, close to 20 percent of people in the United States have GERD. This sensation can sometimes feel similar to the chest pain that people experience during a heart attack or attacks of angina.

Other symptoms of acid reflux and GERD can include a sour or bad taste in the mouth bad breath; tooth decay; difficulty swallowing; nausea and vomiting; or a hoarse voice.

While heart attacks are a life-threatening medical emergency, GERD is not. Therefore, being able to recognize the difference between cardiac and noncardiac (GERD) chest pain would be beneficial.

### SUMMARY OF THE INVENTION

An apparatus or device for testing a patient for distinguishing between symptoms of a heart attack and GERD. The apparatus includes a display configured to display graphical and/or textual information. A saliva specimen holder is configured to collect a saliva sample. A blood specimen holder is configured to collect a blood sample. The blood specimen holder includes a well configured to collect a blood sample, a D-dimer testing zone, and a membrane in fluid communication with the well and the D-dimer testing zone. The membrane is configured to fluidly communicate the blood sample to the D-dimer testing zone. A composition on the membrane in the D-dimer testing zone is configured, when mixed with blood containing a D-dimer protein, to provide an indicator.

A processor is programmed to analyze a collected saliva sample and a blood sample, The processor is programmed to analyze the collected saliva sample to determine whether pepsin is detected in the collected saliva sample. The processor is programmed to send information to the display indicating whether or not pepsin was detected and/or whether or not pepsin was detected above the threshold level. The processor is also programmed to analyze the collected blood sample to determine whether a D-dimer protein is detected in the blood sample.

In an embodiment, the saliva specimen holder is disposable and includes a collection well mounted on a holder. The collection well is configured to collect a suitable amount of a saliva sample from a patient. The apparatus may include a housing with an input port configured to receive the collection well of the saliva specimen holder.

In an embodiment the apparatus includes a housing. The saliva specimen holder may be disposable and include a collection well configured to receive a saliva sample, a first membrane in fluid communication with the collection well and a conjugate pad. The first membrane is configured to fluidly communicate the saliva sample to the conjugate pad. The conjugate pad includes an antibody conjugate configured to combine with pepsin contained in the saliva sample. The conjugate pad may contact a second membrane that is in fluid communication with a pepsin testing zone. The second membrane is configured to fluidly communicate the combined saliva and antibody conjugate to the pepsin testing zone. The second membrane included a first composition at a first location on the membrane that is configured to interact with pepsin in the combined saliva sample and antibody conjugate and provide an indicator when there is pepsin detected in the combined saliva sample and antibody conjugate. A second composition is located at a second location spaced apart from the first location that is configured to interact with the combined saliva sample and antibody conjugate and provide an indicator when the antibody conjugate is detected, The housing may include an input port configured to receive at least a portion of the saliva specimen holder.

The housing may include an imager connected to the processor. The imager may be positioned to monitor the pepsin testing zone when a saliva specimen holder is inserted into the input port and provide image data to the processor. The processor may be programmed to detect the presence of the indicator and provide information to the display indicative of whether or not the indicator has been detected.

In an embodiment, the antibody conjugate is a AuNP-antibody conjugate.

In an embodiment, the blood specimen holder is a disposable cassette, and wherein the housing includes an input port configured to receive at least the portion of the cassette that includes the testing zone. The apparatus may include an imager connected to the processor that is configured to monitor the testing zone in the blood specimen holder when the cassette is inserted into an input port in the housing. The imager provides image data to the processor and the processor is programmed to detect the presence of an indicator in the image data. The processor is programmed to provide information to the display indicative of whether or not the indicator has been detected.

The disposable cassette may be contained in a package with a disposable dropper containing a volume of diluent buffer for mixing with the blood sample.

In an embodiment the composition on the membrane in the D-dimer testing zone is an anti-D-dimer antibody conjugate. The anti-D-dimer antibody may be an anti-D-dimer-gold conjugate.

The indicator may be a colored marking on the membrane.

The threshold level of the pepsin may be approximately 76 ng/mL.

Optionally the membrane in the D-dimer testing zone includes a second composition on the membrane spaced apart from the first composition. The second composition may be configured to provide a second indicator when mixed with a blood sample. The processor may be programmed to analyze data from the imager and to provide information to the display when it detects the second indicator which indicates that a proper blood sample was received.

In an embodiment, the apparatus includes a housing with at least one input port. The saliva specimen holder is a disposable cassette and includes a collection well configured to receive a saliva sample, a first membrane in fluid communication with the collection well and a conjugate pad. The first membrane is configured to fluidly communicate the saliva sample to the conjugate pad, the conjugate pad includes an antibody conjugate configured to combine with pepsin contained in the saliva sample. In an embodiment, the conjugate pad contacts a second membrane that is in fluid communication with a pepsin testing zone. The second membrane is configured to fluidly communicate the combined saliva sample and antibody conjugate to the pepsin testing zone. The second membrane includes a first composition at a first location on the membrane that is configured to interact with pepsin in the combined saliva sample and antibody conjugate and provide an indicator when there is pepsin detected in the combined saliva sample and antibody conjugate. A second composition is located at a second location spaced apart from the first location that is configured to interact with the combined saliva sample and antibody conjugate and provide an indicator when the antibody conjugate is detected. Preferably the input port is configured to receive at least a portion of the disposable cassette.

In this embodiment, the blood sample holder may be a disposable cassette and the housing may include an imager connected to the processor and configured to monitor the pepsin testing zone when the cassette containing the saliva sample is inserted into the at least one input port and to monitor the D-dimer testing zone when the cassette containing the blood sample is inserted into the at least one input port. The imager is configured to send image data to the processor. The processor is programmed to detect the presence of an indicator in the image data and provide information to the display indicative of whether or not the indicator has been detected in the image data.

In an embodiment, an apparatus for testing a patient for distinguishing symptoms of a heart attack and GERD is disclosed where the apparatus includes a housing including a saliva specimen holder configured to collect a saliva sample from a patient, and a blood specimen holder configured to collect a blood sample from the patient. The saliva specimen holder includes a well configured to collect the saliva sample, a conjugate for mixing with the saliva sample, a pepsin testing zone for mixing the combined conjugate and saliva sample with a composition, and at least one membrane in fluid communication with the well and the pepsin testing zone. The at least one membrane is configured to fluidly communicate a collected saliva sample to the conjugate and the combined conjugate and saliva sample to the pepsin testing zone through capillary action. The composition is located on the at least one membrane in the pepsin testing zone and is configured, when mixed with the combined conjugate and saliva sample containing pepsin, to provide a pepsin indicator. The blood specimen holder includes a well configured to collect a blood sample, a D-dimer testing zone, and a membrane configured to fluidly communicate a collected blood sample from the well to the D-dimer testing zone. A composition is located on the membrane in the D-dimer testing zone and is configured, when mixed with blood containing a D-dimer protein, to provide a D-dimer indicator. A display is provided with a window over at least the pepsin testing zone and the D-dimer testing zone.

A method is provided for testing a patient for distinguishing symptoms of a heart attack and GERD. The method includes the steps of collecting a sample of blood with a blood specimen holder; adding a diluent buffer to the collected blood sample to form a diluted blood sample, wherein the diluted blood sample is fluidly communicated by a membrane to a D-dimer testing zone to permit the diluted blood to mix with a composition, the composition configured to provide an indication of the presence of D-dimer protein in the diluted blood sample, and wherein a display indicates if there is a presence of D-dimer protein in the blood sample; collecting a sample of saliva with a saliva specimen holder; analyzing the collected saliva sample to determine the presence of pepsin in the sample of saliva; and displaying information if an amount of pepsin in the collected saliva sample is above a threshold amount.

In an embodiment, the step of analyzing the collected saliva sample involves fluidly communicating the collected saliva sample from a collection well in the saliva specimen holder to a pepsin testing zone; mixing the collected saliva sample with a conjugate, the conjugate configured to interact with pepsin in the collected saliva sample; and mixing the combined saliva sample and conjugate with a composition, the composition configured to provide an indicator of the presence of pepsin in the combined saliva sample and conjugate.

The blood specimen holder and the saliva sample holder may be disposable cassettes, and the method may involve the step of inserting the disposable cassettes into a port on a housing.

The blood specimen holder may be formed on a portion of a housing and the saliva specimen holder may be formed on another portion of the housing. The display may be located on the housing. The step of displaying information may involve indicating the potential for GERD if the presence of D-dimer protein is not detected and the presence of pepsin is detected above a threshold level.

In the method the housing may include an imager configured to monitor the D-dimer testing zone. The method may involve monitoring the D-dimer testing zone for an indicator on the membrane, and monitoring the pepsin testing zone for detecting an indicator on the membrane.

The method may involve the step of analyzing the collected saliva sample is only conducted if there is no indication of D-dimer protein in the collected blood sample.

The foregoing and other features of the invention and advantages of the present invention will become more apparent in light of the following detailed description of the preferred embodiments, as illustrated in the accompanying figures. As will be realized, the invention is capable of modifications in various respects, all without departing from the invention. Accordingly, the drawings and the description are to be regarded as illustrative in nature, and not as restrictive.

### BRIEF DESCRIPTION OF THE DRAWINGS

For the purpose of illustrating the invention, the drawings show a form of the invention which is presently preferred. However, it should be understood that this invention is not limited to the precise arrangements and instrumentalities shown in the drawings.

FIG. 1 is a schematic representation of one embodiment of a device for testing for biomarkers according to the present invention.

FIG. 2A is a schematic drawing of one configuration of a saliva specimen holder.

FIG. 2B is a schematic drawing of another configuration of a saliva specimen holder.

FIG. 2C is a schematic drawing of another configuration of a saliva specimen holder.

FIG. 2D is a cross-sectional side view of the saliva specimen holder of FIG. 20

FIG. 3A is a schematic drawing of one configuration of a blood specimen holder.

FIG. 3B is a cross-sectional side view of the blood specimen holder of FIG. 3A.

FIG. 4 is one embodiment of a method for testing for biomarkers according to the present invention.

FIG, 5 is a schematic representation of another embodiment of a device for testing for biomarkers according to the present invention.

FIG. 5A is a cross-sectional side view of the device of FIG. 5.

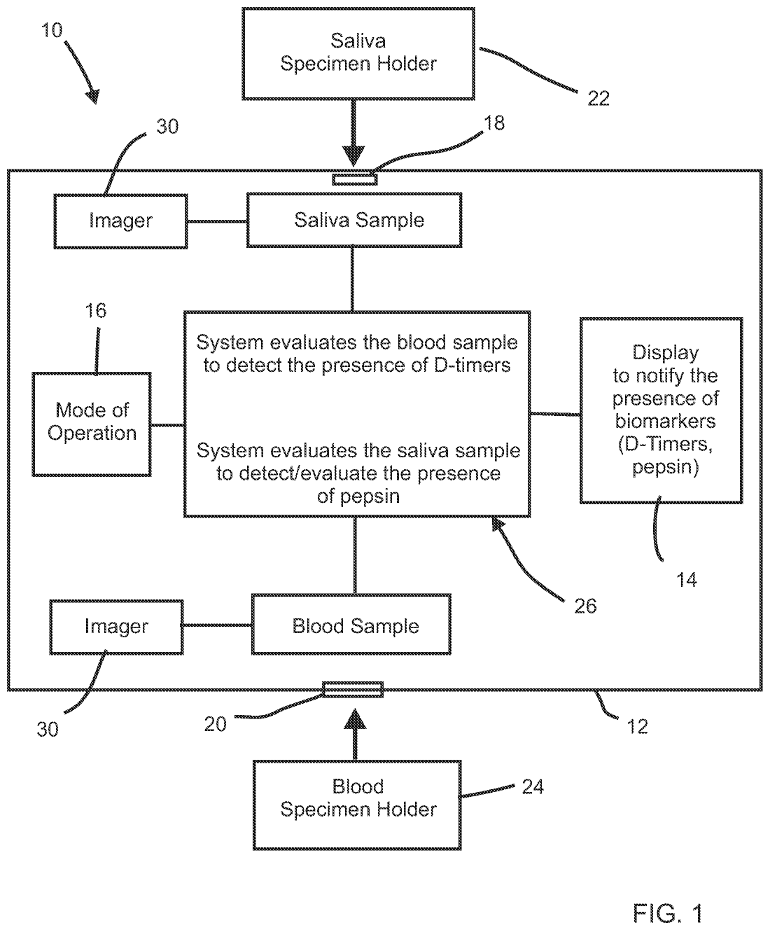

### DESCRIPTION OF THE PREFERRED EMBODIMENTS

Referring to the drawings where preferred embodiments of the invention are depicted, the present invention, in at least one embodiment, is directed to a device 10 for detecting and analyzing biomarkers. The device 10, which may comprise a kit, includes a housing 12 with a display 14 configured to display graphical and/or textual information, and, in one or more embodiments, one or more user activated inputs keys 16, such as touch pads. If the display is a touch screen, the user input keys would be incorporated into the display 14. The housing 10 includes at least one, and in the illustrated embodiment two sample input ports 18, 20 configured to receive a disposable saliva specimen holder 22 and a blood specimen holder 24 as will be discussed in more detail below.

A processor 26 can be mounted inside the housing 10. The processor 26 is programmed to analyze (e.g.., evaluate) collected saliva and blood samples. For example, the processor 26 can be programmed to analyze a sample of saliva collected by (e.g., contained on or placed in) the saliva specimen holder 22, and can be programmed to analyze a blood sample collected by (e.g., contained on or placed in) the blood specimen holder 24.

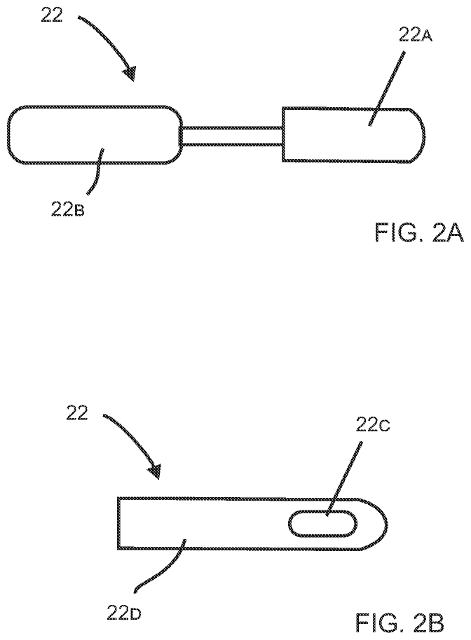

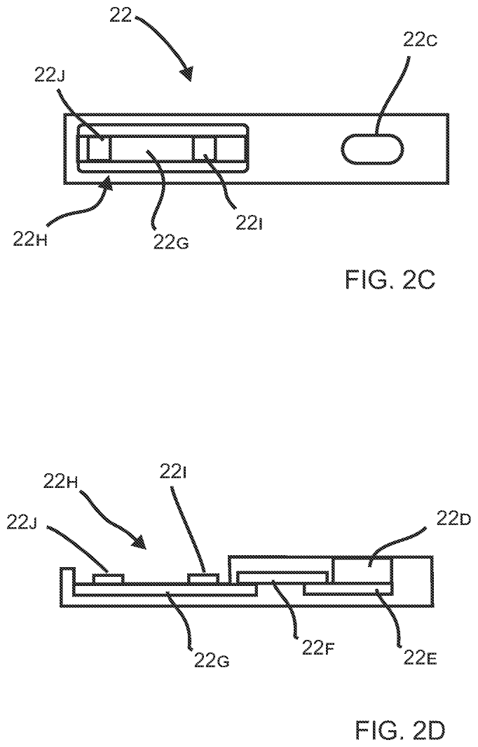

Referring to FIG. 2A, the saliva specimen holder 22 is configured to collect (capture or receive) a saliva sample from a patient. In one embodiment, the saliva specimen holder is a cotton pad 22A on a holder 22B that a user can insert into the mouth of a patient to collect a sufficient amount of saliva, for example, about 1 mL. The cotton pad of the saliva specimen holder 22 is then inserted into one of the sample input ports 18 for analysis. Alternatively, as shown in FIG. 2B, the saliva specimen holder 22 could be a cassette type holder that includes a collection well 22c for the patient to spit into. The collection well is mounted on a holder 22D. After the patient spits a sufficient volume of saliva into the well 22c, the user inserts the well 22c into the sample input port 18 for analysis.

The processor 26 can be configured to analyze the saliva sample (e.g., contained on or placed in) the saliva specimen holder 22 to determine whether a pepsin biomarker is detected in the saliva indicative of the existence of pepsin in the saliva and, if so, to determine the amount of pepsin in the saliva or whether the detected pepsin is above a threshold level, such as 76 ng/mL. The processor 26 is programmed to send information (e.g., a signal) to the display 14 for displaying information indicating whether or not pepsin was detected and/or whether or not pepsin was detected above the threshold level.

There are several ways known in the art for analyzing saliva to detect pepsin. In one embodiment, the housing includes micro centrifuge or other device to remove insoluble materials. A migration buffer is then added to the sample and the combination is applied to a test strip, such as the Peptest test strip developed by RDBiomed, Cottingham UK. The test strip includes a nitrocellulose membrane with two spaced apart types of monoclonal antibodies against pepsin. One antibody is tagged with a colorimetric marker and becomes soluble when a test sample reaches it. The other antibody is immobilised onto the nitrocellulose membrane at the test line. Any pepsin in the sample is bound at the test line by the two antibodies, and a colored line becomes visible. The device can include an imager, such as a video camera or other conventional imaging device, that monitors the location where the antibody that includes the colorimetric marker is located. The imager provides image data of the test strip. The processor 26 is programmed to detect the presence of the calorimetric marker, and to send a signal or other information indicative of the present of pepsin to the display 14.

In an embodiment, the saliva specimen holder 22 includes most or all of the components necessary for conducting the analysis in a lateral flow pepsin testing device or cassette. See, for example, Young Ju Lee et al., “Optimization of Saliva Collection and mmunochromatographic Detection of Salivary Pepsin for Point-of-Care Testing of Laryngopharyngeal Reflux”, Sensors 20(1):325, January 2020 (the “Lee reference”), the disclosure of which is incorporated herein by reference in its entirety, that discloses a lateral flow pepsin immunochromatographic strip which can be used in the present invention. Referring to FIGS. 2C and 2D, the saliva specimen holder 22 includes a collection well 22_{C} mounted on or located in a holder 22_{D}. A first membrane 22E is in fluid communication with the well and a conjugate pad 22_{F}. The first membrane is, in one embodiment, a nitrocellulose membrane that is configured to fluidly communicate the saliva sample to the conjugate pad 22_{F}, such as through capillary action. The conjugate pad 22_{F} includes an antibody conjugate, which in one embodiment is a nanoparticle (AuNP)-antibody conjugate, configured to combine (e.g., capture or mix with) pepsin in the saliva sample. A second membrane 22_{G} is in fluid communicate with the conjugate pad 22_{F} and a pepsin testing zone 22_{H}. The second membrane 22_{G} is, in one embodiment, a nitrocellulose membrane that is configured to fluidly communicate the combined saliva sample and conjugate to the pepsin testing zone 22_{H} e.g., such as through capillary action. The second membrane 22_{G} includes a first antibody (monoclonal) composition that is configured to interact with pepsin contained in the combined saliva sample and conjugate, and an indicator, such as a colorimetric marker, that is activated by the interaction. The colorimetric marker may be configured to be activated only when the amount of pepsin in the combined saliva sample and conjugate is above a threshold level that is indicative of GERD, such as 76 ng/mL. The first antibody composition is located at a first location 22_{I} within the pepsin testing zone 22_{H}. A second antibody (monoclonal) composition with an indicator (e.g., a calorimetric marker) may be included at a second location 22_{J} spaced apart from the first location 22_{I} and further from the sample well. The second antibody composition is configured to interact with the AuNP-antibody conjugate such that the indicator is activated by the interaction. Suitable first and second antibodies are disclosed in the Lee reference.

if the AuNP-antibody conjugate has combined with any pepsin in the saliva sample it will bind with the first composition at the first location 22_{I} and trigger its indicator (e.g., its calorimetric marker). The remaining AuNP-antibody conjugate will proceed to the second location 22_{J} and interact with the second composition and trigger its indicator e.g., its calorimetric marker). The presence of the indictor at the first location 22_{I} indicates the presence of pepsin in saliva above a first threshold level. The presence of the indicator at the second location 22_{J} indicates that the analysis was properly conducted.

A digital imager 30 may be positioned inside the housing 12 so as to monitor the pepsin testing zone 22_{H} for detecting the appearance of an indicator (e.g., detect the presence of a color marker) at the first and second locations. The digital imager may send image data to the processor 26 for determining the presence of the indicators,

It is contemplated that it might be necessary to add a migration buffer to the saliva sample in order to facilitate the capillary action. Suitable migration buffers are well known. A suitable volume of the buffer could be included in a vial in a prepackaged kit that contains a disposable saliva specimen holder 22. After the patient spits into the saliva sample well, the user would add the preset amount of buffer from the vial into the sample well. Alternatively, the housing 12 may contain a reservoir of buffer that is automatically added after the saliva specimen holder 22 is inserted. It is also contemplated that the buffer could be stored in the first membrane 22_{E} and/or the conjugate pad 22_{F} in known ways, such as by pre-treatment and drying.

The housing 12 may include a contact (e,g., pressure pad or similar component) configured to detect when the saliva specimen holder 22 is properly inserted into the input port at which point it activates the imager 30. Alternatively, a user input key 16 on the housing 12 could be depressed to activate the imager 30.

The processor 26 can be programmed to analyze the saliva sample for detecting pepsin as discussed above. If pepsin is detected above a threshold level, for example, the imager 30 detects the color indicator at the first location 22_{I} indicative of the presence of pepsin above the threshold level, the processor 26 sends a suitable message on the display 14.

The processor 26 can be programmed to analyze a blood sample collected by a blood specimen holder 24 to determine whether a D-dimer protein biomarker is detected in the blood sample. The D-dimer protein is a protein fragment that develops in blood when blood clots are actively forming and breaking down in the blood. More specifically, when a blood vessel or tissue is injured and begins to bleed, a process called hemostasis is initiated by the body to create a blood clot to limit and eventually stop the bleeding. This process produces threads of a protein called fibrin, which crosslink together to form a fibrin net. That net, together with platelets, helps hold the forming blood clot in place at the site of the injury until it heals. Once the area has had time to heal and the clot is no longer needed, the body uses an enzyme called plasmin to break the clot (thrombus) into small pieces so that it can be removed. The fragments of the disintegrating fibrin in the clot are called fibrin degradation products (FDP), which consist of variously sized pieces of crosslinked fibrin. One of the final fibrin degradation products produced is D-dimer, which is a small protein fragment present in the blood after a blood clot is degraded by fibrinolysis. It is so named because it contains two D fragments of the fibrin protein joined by a cross-link.

Normally, the amount of detectable D-dimer protein in a blood sample is below a threshold level generally indicative of a low amount of D-dimer protein. When the amount of D-dimer protein detected is above the threshold level, that is indicative that blood clots are actively forming in the body and that the body is working to break down the clots, i.e., there is significant formation and breakdown of fibrin clots in the body. This typically happens during a heart attack.

The processor 26 analyzes the blood sample collected by the blood specimen holder 24 to determine whether D-dimer proteins are detected in the blood sample and, if so, to determine the amount of D-dimer proteins present or whether the detected D-dimers are above a threshold level. For example, in one embodiment the threshold level of D-dimer proteins is above 0.5 μg mL⁻¹. It is anticipated that the threshold level used would vary depending on the analysis used.

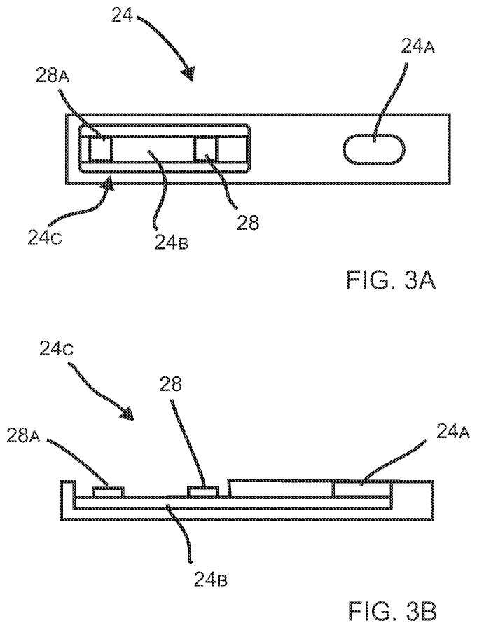

In one embodiment, the D-dimer protein analysis is provided as follows with reference to FIGS. 3A and 3B. The blood specimen holder 24 can be a cassette that includes a well 24_{A} that is configured to collect a blood sample (e.g., for a patient). A membrane 24_{B} is in fluid communication with (e.g., located at the bottom of) the well 24_{A} and a testing zone 24c. A drop of diluent buffer can be added to the well 24_{A} to mix with the blood sample. In one embodiment, the blood specimen holder 24 and a disposable vial containing a predetermined amount of diluent buffer are provided in a single use package that the user opens when testing a patient. Alternatively, the diluent buffer could be stored on the membrane, such as be preapplication.

The diluted blood sample is fluidly communicated by (e.g., moves on or is transported by) the membrane, such as through capillary action, to the testing zone 24_{C} where the diluted blood sample reacts with a composition (e.g., conjugate) on the membrane. The conjugate can be an anti-D-dimer antibody, such as anti-D-dimer antibody-gold conjugate. If D-dimer is present in the blood sample, an indicator 28, such as a colored marking or other visual indicator, will form from the mixing of the anti-D-dimer antibody, D-dimer from the specimen and the antibody on the membrane.

An imager 30, such as a video camera or other conventional imaging device, monitors the testing zone 24_{C}. The imager 30 provides image data of the testing zone. The processor 26 is programmed to detect the presence of the indicator 28, and to send information, such as a signal, indicative of the presence of D-dimer proteins to the display 14.

In one embodiment, the second specimen holder 24 is a Biosynex® D-dimer cassette sold by Biosynex S A, Illkirch-Graffenstaden, France, which includes the sample well 24_{A}, membrane 24_{B} and testing zone 24_{C}. In this embodiment, the imager 30 would be inside the device 10 and the cassette would be inserted into the sample input port 20, which positions the cassette testing zone 24_{C} below the imager 30. One feature of the Biosynex® D-dimer cassette that is useful is that the testing zone 24_{C} includes two indicators, one indicator 28_{A} is configured to indicate that the blood sample was properly analyzed and the other indicator 28 is configured to indicate whether or not a certain level of D-dimer proteins are present in the sample. In this case, the processor 26 is programmed to analyze the images received from the imager 30 for detecting the presence of indicators 28, 28A in two different locations of the testing zone 24_{C}. The processor 26 can display information related to the analysis, such as that the blood sample was properly detected, and whether or not D-dimer is present in the sample. This will reduce any incomplete information such as if the blood sample did not properly saturate the testing zone 24_{C}.

The housing 12 can also include a contact (e.g., pressure pad or similar component) configured to detect when the blood specimen holder 24 is properly inserted into the input port at which point it activates the imager 30. Alternatively, a user input key 16 on the housing 12 could be depressed to activate the imager 30.

The second specimen holder 24 may include a built-in needle or other sharp implement configured to puncture a user's finger to facilitate the collection of a blood sample.

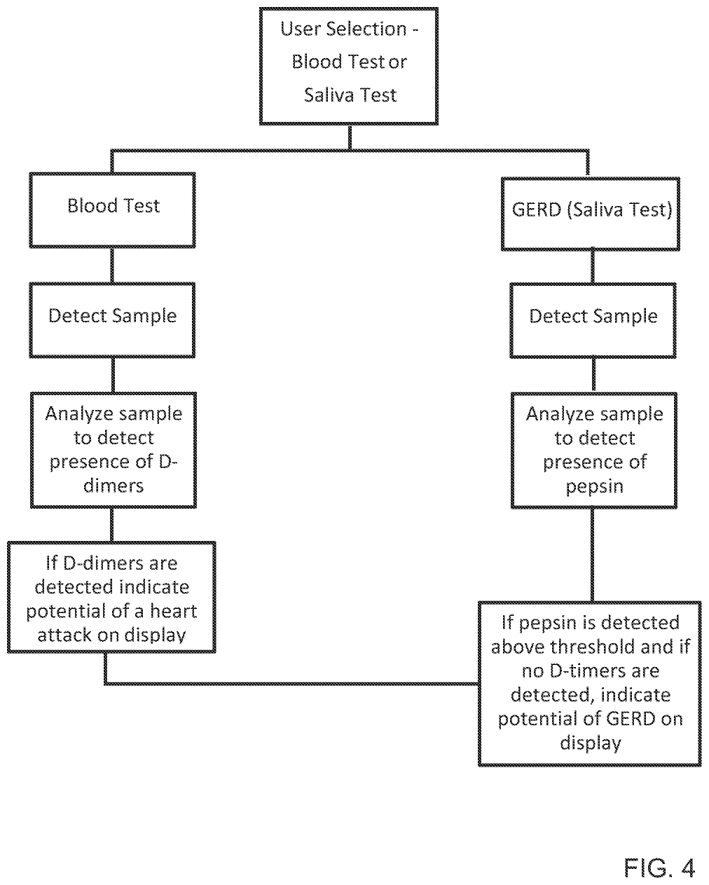

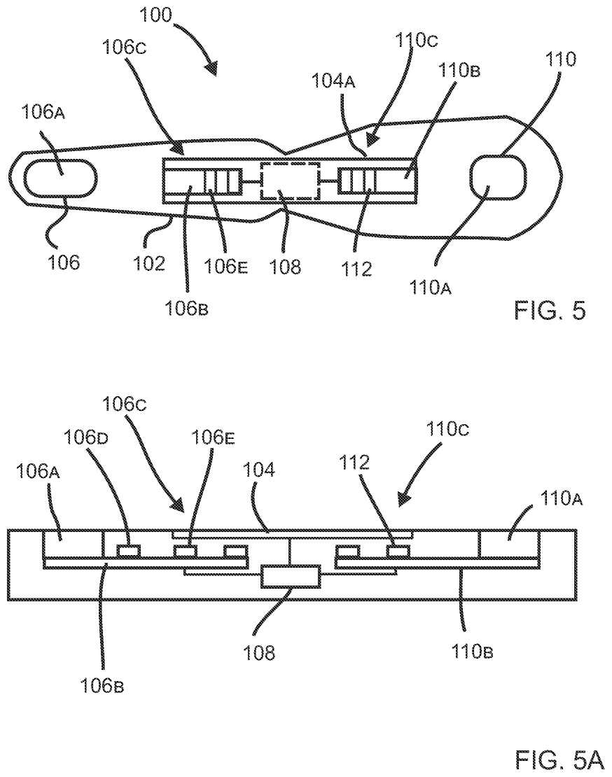

The invention will be better understood with respect to its operation and reference to FIG. 4. A user activates the device 10 and has the option to select the testing mode desired, e.g., blood sample testing or saliva sample testing, using the input keys 16. It is also contemplated that the system could automatically select blood sample testing in order to first detect whether D-dimer proteins are present in the patient's blood sample. The display 14 may display information to facilitate the proper collection of blood, such as explaining the steps that the user should follow. The user selects a blood specimen holder 24 and pricks the finger of the patient. A drop of blood is placed in the well 24_{A} and a drop of diluent buffer is added. The user then inserts the blood specimen holder 24 into the appropriate input port 20 on the housing 12. The user may then activate the device 10 to begin the analysis or the inserting of the blood specimen holder 24 may automatically commence the analysis as discussed above.

The imager 30 analyzes the testing zone 24_{C} for detecting an indicator 28. After a prescribed period of time, if no indicator 28 is detected, the processor 26 sends a message to the display to inform the user that no D-dimer protein was detected. The processor can also display information indicating that the blood sample was properly analyzed, for example if a second indicator 28A is provided, when the diluted blood flows along the membrane to the second indicator 28A and activates it as discussed above, that indicates that a sufficient amount of blood has been received and has made it to the indicator 28. If an indicator 28 is detected, the processor 26 sends a message to the display to inform the user that D-dimer proteins were detected in the blood sample.

If D-dimer proteins are detected (or the amount detected are above a threshold amount) in the blood sample, it is an indication that the patient may have been or may be suffering from a heart attack. As such, the processor 26 may display a message that the patient should be immediately taken for medical assistance. In this case, the saliva sample testing may not be needed.

If no D-dimer proteins are detected (or the amount detected are below a threshold amount) in the blood sample, the processor 26 may inform the user via the display 14 to conduct a saliva sample test. The user would then collect a sufficient sample of saliva using the saliva specimen holder 22. The saliva specimen holder 22 is then inserted into the appropriate input sample port 18. The saliva sample is then analyzed for the existence of pepsin in the saliva sample. If the processor 26 determines that pepsin exists above a threshold level, it sends a signal to the display 14 to inform the user that the patient may be experiencing GERD. If the processor 26 determines that there is no pepsin in the saliva or if the detected pepsin is below a threshold level, the processor sends a signal to the display 14 to inform the user that the patient may not be experiencing GE RD.

Referring to AGS. 5 and 5A, an alternate embodiment of the device 100 is shown. In this embodiment, the device is a disposable, single-use testing kit. The device includes a housing 102 with a display 104, A saliva specimen holder 106 is located at one end and includes a well 106_{A} for collecting (e.g., capturing) a suitable amount of saliva. A membrane 106_{B} is located at the bottom of the well 106_{A} and extends in the housing 102 from the well 106_{A} toward a pepsin testing zone 106_{C}. As discussed above, the membrane 106_{B} preferably includes a conjugate 106_{D} and one or more compositions 106_{E} on the membrane in the testing zone 110_{C}, the compositions are configured to provide an indicator if pepsin is detected as discussed above. In one embodiment, the display 104 may include a window (e.g., an aperture) 104_{A} over the pepsin testing zone 110_{C} which permits the user to see the presence of an indicator.

On the opposite side of the housing 102 is a blood specimen holder 110 which includes a well 110_{A} for collecting (capturing) a suitable amount of blood, A membrane 110_{B} is located at the bottom of the well 110A and extends in the housing 102 from the well 110_{A} toward the testing zone 110_{C}. Similar to the Biosynex® D-dimer cassette, the membrane 110_{B} in the testing zone 110_{C} may include an anti-D-dimer antibody such that it provides an indicator 112 if D-dimers are detected as discussed above.

In one embodiment, the display 104 may include a window (e.g., an aperture) 104_{A} over the testing zones 106_{C} and 110_{C} which permits the user to see the presence of an indicator 112. Alternatively, the display 104 could be an graphical display that provides an alphanumeric output.

A processor 108 may be positioned below or adjacent to the display 104. The processor 108 may include a computer processing chip programmed to provide analysis as discussed above. The processor 108 may be programmed to provide a result to the display 104 from each analysis.

Alternatively, instead of depicting the results in the display 104 of both the saliva test and the blood test, it is contemplated that the processor 108 may be programmed to conduct both the saliva test analysis and the blood test analysis described above and then indicate on the display 104 a result, such as a YES/NO, as to whether the testing is indicative of symptoms of a heart attack or GERD.

While the above description discloses embodiments including separate input ports and imaging devices for the saliva sample holder and the blood sample holder, it is contemplated that there could be a single input port and a single imaging device, the user would select the applicable testing to be conducted (by means of the user input keys) based on which sample holder is inserted into the input port.

The description and illustrations of one or more aspects provided in this application are not intended to limit or restrict the scope of the disclosure as claimed in any way. The aspects, examples, and details provided in this application are considered sufficient to convey possession and enable others to make and use the best mode of claimed disclosure. The claimed disclosure should not be construed as being limited to any aspect, example, or detail provided in this application. Regardless of whether shown and described in combination or separately, the various features (both structural and methodological) are intended to be selectively included or omitted to produce an embodiment with a particular set of features. Having been provided with the description and illustration of the present application, one skilled in the art may envision variations, modifications, and alternate aspects falling within the spirit of the broader aspects of the general inventive concept embodied in this application that do not depart from the broader scope of the claimed disclosure.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening.

The recitation of ranges of values herein are merely intended to serve as a shorthand method of referring individually to each separate value falling within the range, unless otherwise indicated herein, and each separate value is incorporated into the specification as if it were individually recited herein.

The use of directions, such as forward, rearward, top and bottom, upper and lower are with reference to the embodiments shown in the drawings and, thus, should not be taken as restrictive. Reversing or flipping the embodiments in the drawings would, of course, result in consistent reversal or flipping of the terminology.

It will be apparent to those skilled in the art that various modifications and variations can be made to the present invention without departing from the spirit and scope of the invention. There is no intention to limit the invention to the specific form or forms disclosed, but on the contrary, the intention is to cover all modifications, alternative constructions, and equivalents falling within the spirit and scope of the invention, as defined in the appended claims. Thus, it is intended that the present invention cover the modifications and variations of this invention provided they come within the scope of the appended claims and their equivalent.

The system or systems described herein may be implemented on any form of computer or computers and the algorithms and programs may be implemented as dedicated applications or in client-server architectures, including a web-based architecture, and can include functional programs, codes, and code segments. The computer system of the present invention may include a software program be stored on a computer and/or storage device (e.g., mediums), and/or may be executed through a network. The computer steps may be implemented through program code or program modules stored on a storage medium.

The computer processes herein may be described in terms of various processing steps. Such processing steps may be realized by any number of hardware and/or software components that perform the specified functions. For example, the described embodiments may employ various integrated circuit components, e.g., memory elements, processing elements, logic elements, look-up tables, and the like, which may carry out a variety of functions under the control of one or more microprocessors or other control devices. Similarly, where the elements of the described embodiments are implemented using software programming or software elements the invention may be implemented with any programming or scripting language such as C, C++, Java, assembler, or the like, with the various algorithms being implemented with any combination of data structures, objects, processes, routines or other programming elements. Functional aspects may be implemented in algorithms that execute on one or more processors. Furthermore, the embodiments of the invention could employ any number of conventional techniques for electronics configuration, signal processing and/or control, data processing and the like. The words “mechanism” and “element” are used broadly and are not limited to mechanical or physical embodiments, but can include software routines in conjunction with processors, etc.

The particular implementations shown and described herein are illustrative examples of the invention and are not intended to otherwise limit the scope of the invention in any way. For the sake of brevity, conventional electronics, control systems, software development and other functional aspects of the systems (and components of the individual operating components of the systems) may not be described in detail.

Finally, the steps of all methods described herein are performable in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. Numerous modifications and adaptations will be readily apparent to those skilled in this art without departing from the spirit and scope of the invention.

1. An apparatus for testing a patient for distinguishing symptoms of a heart attack and GERD, the apparatus comprising:
a display configured to display graphical and/or textual information;
a saliva specimen holder configured to collect a saliva sample;
a blood specimen holder configured to collect a blood sample, the blood specimen holder including a well configured to collect a blood sample, a D-dimer testing zone, and a membrane in fluid communication with the well and the D-dimer testing zone, the membrane configured to fluidly communicate the blood sample to the D-dimer testing zone, wherein a composition on the membrane in the D-dimer testing zone is configured, when mixed with blood containing a D-dimer protein, to provide an indicator; and
a processor programmed to analyze a collected saliva sample and a blood sample, the processor programmed to analyze the collected saliva sample to determine whether pepsin is detected in the collected saliva sample, the processor programmed to send information to the display indicating whether or not pepsin was detected and/or whether or not pepsin was detected above the threshold level, and the processor programmed to analyze the collected blood sample to determine whether a D-dimer protein is detected in the blood sample.

2. The apparatus of claim 1, further comprising a housing and wherein the saliva specimen holder is disposable and comprises a collection well mounted on a holder, the collection well configured to collect a suitable amount of a saliva sample from a patient; and wherein the housing includes an input port configured to receive the collection well of the saliva specimen holder.

3. The apparatus of claim 1, further comprising a housing and wherein the saliva specimen holder is disposable and comprises a collection well configured to receive a saliva sample, a first membrane in fluid communication with the collection well and a conjugate pad, the first membrane configured to fluidly communicate the saliva sample to the conjugate pad, the conjugate pad including an antibody conjugate configured to combine with pepsin contained in the saliva sample, the conjugate pad contacting a second membrane that is in fluid communication with a pepsin testing zone, the second membrane configured to fluidly communicate the combined saliva sample and antibody conjugate to the pepsin testing zone, the second membrane including a first composition at a first location on the membrane that is configured to interact with pepsin in the combined saliva sample and antibody conjugate and provide an indicator when there is pepsin detected in the combined saliva sample and antibody conjugate; and a second composition at a second location spaced apart from the first location that is configured to interact with the combined saliva sample and antibody conjugate and provide an indicator when the antibody conjugate is detected; and wherein the housing includes an input port configured to receive at least a portion of the saliva specimen holder.

4. The apparatus of claim 3, wherein the housing includes an imager connected to the processor, the imager positioned to monitor the pepsin testing zone when a saliva specimen holder is inserted into the input port and provide image data to the processor;
and wherein the processor is programmed to detect the presence of the indicator, the processor programmed to provide information to the display indicative of whether or not the indicator has been detected.

5. The apparatus of claim 4, wherein the antibody conjugate is a AuNP-antibody conjugate.

6. The apparatus of claim 1, further comprising a housing and wherein the blood specimen holder is a disposable cassette, and wherein the housing includes an input port configured to receive at least the portion of the cassette that includes the testing zone.

7. The apparatus of claim 6, further comprising an imager connected to the processor, the imager configured to monitor the testing zone when a cassette is inserted into the input port and provide image data to the processor, and wherein the processor is programmed to detect the presence of the indicator, the processor programmed to provide information to the display indicative of whether or not the indicator has been detected.

8. The apparatus of claim 6, wherein the disposable cassette is contained in a package with a disposable dropper, the disposable dropper containing a volume of diluent buffer for mixing with the blood sample.

9. The apparatus of claim 1, wherein the composition is an anti-D-dimer antibody conjugate.

10. The apparatus of claim 9, wherein the anti-D-dimer antibody is an anti-D-dimer-gold conjugate.

11. The apparatus of claim 1, wherein the indicator is a colored marking on the membrane.

12. The apparatus of claim 1, wherein the threshold level of the pepsin is approximately 76 ng/mL.

13. The apparatus of claim 1, further comprising a second composition on the membrane spaced apart from the first composition, the second composition configured to provide a second indicator when mixed with blood, and wherein the processor is programmed to analyze data from an imager and to provide information to the display when it detects the second indicator which indicates that a proper blood sample was received.

14. The apparatus of claim 1, further comprising a housing that includes at least one input port;
wherein the saliva specimen holder is a disposable cassette and includes a collection well configured to receive a saliva sample, a first membrane in fluid communication with the collection well and a conjugate pad, the first membrane configured to fluidly communicate the saliva sample to the conjugate pad, the conjugate pad including an antibody conjugate configured to combine with pepsin contained in the saliva sample, the conjugate pad contacting a second membrane that is in fluid communication with a pepsin testing zone, the second membrane configured to fluidly communicate the combined saliva sample and antibody conjugate to the pepsin testing zone, the second membrane including a first composition at a first location on the membrane that is configured to interact with pepsin in the combined saliva sample and antibody conjugate and provide an indicator when there is pepsin detected in the combined saliva sample and antibody conjugate; and a second composition at a second location spaced apart from the first location that is configured to interact with the combined saliva sample and antibody conjugate and provide an indicator when the antibody conjugate is detected; and wherein the input port is configured to receive at least a portion of the disposable cassette;
wherein the blood sample holder is a disposable cassette; and
wherein the housing includes an imager connected to the processor, the imager configured to monitor the pepsin testing zone when the cassette containing the saliva sample is inserted into the at least one input port and to monitor the D-dimer testing zone when the cassette containing the blood sample is inserted into the at least one input port, the imager configured to send image data to the processor, and wherein the processor is programmed to detect the presence of an indicator in the image data, the processor programmed to provide information to the display indicative of whether or not the indicator has been detected in the image data.

15. An apparatus for testing a patient for distinguishing symptoms of a heart attack and GERD, the apparatus comprising:
a housing including a saliva specimen holder configured to collect a saliva sample from a patient, and a blood specimen holder configured to collect a blood sample from the patient;
the saliva specimen holder including a well configured to collect the saliva sample, a conjugate for mixing with the saliva sample, a pepsin testing zone for mixing the combined conjugate and saliva sample with a composition, and at least one membrane in fluid communication with the well and the pepsin testing zone, the at least one membrane configured to fluidly communicate a collected saliva sample to the conjugate and the combined conjugate and saliva sample to the pepsin testing zone through capillary action, wherein the composition is located on the at least one membrane in the pepsin testing zone and is configured, when mixed with the combined conjugate and saliva sample containing pepsin, to provide a pepsin indicator;
the blood specimen holder including a well configured to collect a blood sample, a D-dimer testing zone, and a membrane configured to fluidly communicate a collected blood sample from the well to the D-dimer testing zone, wherein a composition is located on the membrane in the D-dimer testing zone and is configured, when mixed with blood containing a D-dimer protein, to provide a D-dimer indicator; and
a display including a window over at least the pepsin testing zone and the D-dimer testing zone.

16. A method for testing a patient for distinguishing symptoms of a heart attack and GERD, the method comprising:
collecting a sample of blood with a blood specimen holder;
adding a diluent buffer to the collected blood sample to form a diluted blood sample, wherein the diluted blood sample is fluidly communicated by a membrane to a D-dimer testing zone to permit the diluted blood to mix with a composition, the composition configured to provide an indication of the presence of D-dimer protein in the diluted blood sample, and wherein a display indicates if there is a presence of D-dimer protein in the blood sample;
collecting a sample of saliva with a saliva specimen holder;
analyzing the collected saliva sample to determine the presence of pepsin in the sample of saliva; and
displaying information if an amount of pepsin in the collected saliva sample is above a threshold amount.

17. The method according to claim 16, wherein the step of analyzing the collected saliva sample comprises the steps of:
fluidly communicating the collected saliva sample from a collection well in the saliva specimen holder to a pepsin testing zone;
mixing the collected saliva sample with a conjugate, the conⱼugate configured to interact with pepsin in the collected saliva sample; and
mixing the combined saliva sample and conjugate with a composition, the composition configured to provide an indicator of the presence of pepsin in the combined saliva sample and conjugate.

18. The method according to claim 16, wherein the blood specimen holder and the saliva sample holder are a disposable cassettes, and wherein the method further comprises the step of inserting the disposable cassettes into a port on a housing.

19. The method according to claim 16, wherein the blood specimen holder is formed on a portion of a housing; wherein the saliva specimen holder is formed on another portion of the housing; wherein the display is located on the housing; and wherein the step of displaying information involves indicating the potential for GERD if the presence of D-dimer protein is not detected and the presence of pepsin is detected above a threshold level.

20. The method according to claim 17, wherein the housing includes an imager configured to monitor the D-dimer testing zone; and wherein the method involves the steps of: monitoring the D-dimer testing zone for an indicator on the membrane, and monitoring the pepsin testing zone for detecting an indicator on the membrane.

21. The method according to claim 16, wherein the step of analyzing the collected saliva sample is only conducted if there is no indication of D-dimer protein in the collected blood sample.
